(19) United States
(12) Patent Application Publication
(10) Pub. No.: US 2024/0350775 A1
(43) Pub. Date: Oct. 24, 2024

(54) INTRODUCER SHEATH ASSEMBLIES AND RELATED SYSTEMS AND METHODS

(71) Applicant: Merit Medical Systems, Inc., South Jordan, UT (US)

(72) Inventors: Michael Dean Haslam, Sandy, UT (US); Jim Mottola, West Jordan, UT (US); Nicole Willardson, Herriman, UT (US); Richard D. Jenkins, Kennett Square, PA (US)

(21) Appl. No.: 18/643,374

(22) Filed: Apr. 23, 2024

Related U.S. Application Data
(60) Provisional application No. 63/497,843, filed on Apr. 24, 2023.

(51) Int. Cl. A61M 25/06; A61M 25/00; A61M 39/06

(52) U.S. Cl. CPC A61M 25/0668; A61M 25/0097; A61M 39/0613; A61M 2039/062; A61M 2039/0626; A61M 2039/0633; A61M 2205/0216

(57) ABSTRACT

The present disclosure illustrates introducer sheath assemblies, and related systems and methods of use. An introducer sheath assembly may include a hub, a cap, an elastomeric member, and a sheath. The elastomeric member may have an hourglass shape disposed in a proximal portion of a lumen of the hub and that is deformable responsive to movement of the cap.

### RELATED APPLICATIONS

This application claims priority to U.S. Provisional Application No. 63/497,843, filed on Apr. 24, 2023 and titled “INTRODUCER SHEATH ASSEMBLIES AND RELATED SYSTEMS AND METHODS,” which is hereby incorporated by reference in its entirety.

### TECHNICAL FIELD

The present disclosure relates generally to medical sheaths including introducer sheaths and methods to manufacture introducer sheaths.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a perspective view of an introducer sheath assembly and a dilator, according to one embodiment.

FIG. 1B is an exploded view of the introducer sheath assembly of FIG. 1A.

FIG. 1C is a perspective view of a proximal portion of the introducer sheath assembly of FIG. 1A.

FIG. 2 is a cross-sectional view of a portion of the dilator of FIG. 1A.

FIG. 3A is a cross-sectional view of the proximal portion of the introducer sheath assembly taken through line 3-3 in FIG. 1C with the dilator removed.

FIG. 3B is an exploded view of the proximal portion of the introducer sheath assembly in FIG. 1C with the dilator removed.

FIG. 4A is a cross-sectional view of the proximal portion of the introducer sheath assembly taken through line 4-4 in FIG. 1C with the dilator removed.

FIG. 4B is a cross-sectional view of a portion of an elastomeric member that may be used in the introducer sheath assembly of FIG. 1A, according to one embodiment.

FIG. 5A is a perspective view of an introducer sheath assembly, dilator, and side arm catheter, according to one embodiment.

FIG. 5B is a perspective view of an introducer sheath assembly, dilator, and side arm catheter, according to one embodiment.

FIG. 6A is an exploded view of a proximal portion of the introducer sheath assembly of FIG. 5A.

FIG. 6B is a cross-sectional view of the proximal portion of the introducer sheath assembly of FIG. 5A.

FIGS. 7A-7C are cross-sectional views of an elastomeric member of the introducer sheath assembly of FIG. 5A at various stages of deformation.

FIG. 8 is a flow diagram of a method of inserting an elongate member into a subject, according to one embodiment.

FIG. 9 is a flow diagram of a method of inserting an elongate member into a subject, according to one embodiment.

### DETAILED DESCRIPTION

This disclosure describes introducer sheath assemblies (e.g., introducer sheaths or medical sheaths) having lumens or bores adapted for use with larger-sized medical instruments. To facilitate the exchange of medical tools and fluids, the introducer sheath includes a hub configured to remain exterior to the patient's skin. A practitioner may introduce guidewires, catheters, stents, balloons, and other articles and/or materials to be introduced into the patient through the hub. A valve or a seal may maintain hemostasis of the introducer sheath while allowing a medical instrument to be introduced through the hub into the chamber. The sheath shaft is inserted into the vasculature of the patient, and provides through the skin to the vasculature.

This disclosure describes introducer sheath assemblies (e.g., medical sheaths) having lumens or bores adapted for use with larger-sized medical instruments. In some embodiments, the introducer sheath assemblies of this disclosure may be configured for use with 9 F to 20 F elongate members of medical instruments and/or 14 F to 20 F elongate members of medical instruments. For example, at least one, some, or all embodiments of introducer sheath assemblies of this disclosure may be configured for use with 9 F to 20 F or 14 F to 20 F dilators, dialysis catheters, or other catheters.

In some embodiments, introducer sheath assemblies of this disclosure may be configured to be split apart after insertion of the elongate member for removal of the introducer sheath assembly without disruption of the elongate member. For example, one or more (e.g., all) of a hub, an elastomeric member (e.g., valve), cap, and/or sheath of an introducer sheath of this disclosure may be configured to be spilt apart after insertion of the elongate member during use.

In some embodiments, introducer sheath assemblies of this disclosure may be configured to include an elastomeric member that functions as an active valve within the introducer sheath assembly. For example, the elastomeric member of embodiments of introducer sheath assemblies of this disclosure may be selectively adjustable to increase, decrease, and/or close a lumen extending through the elastomeric member. This configuration of the elastomeric member may allow a single sized elastomeric member (and introducer sheath assembly) to be used with a variety of sizes of elongate members of medical instruments. In some embodiments, an elastomeric member of an introducer sheath assembly of this disclosure may be adjustable or otherwise configured to form a liquid-tight seal around any medical instrument having a size of 9 F to 16 F or 9 F to 20 F.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. As used herein, the distal end of a device or component is the end of the component that is furthest from the physician during ordinary use. The proximal end refers to the opposite end, or the end nearest the physician during ordinary use. For example, the proximal end of an introducer sheath used in minimally invasive vascular treatment is the end accessible to a practitioner during use, while the distal end is disposed within a patient's vascular system when the sheath is placed into such a patient.

An assembler may be any person, system, or machine used in the manufacture of the introducer sheaths.

Embodiments may be understood by reference to the drawings, wherein like parts are designated by like numerals throughout. The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

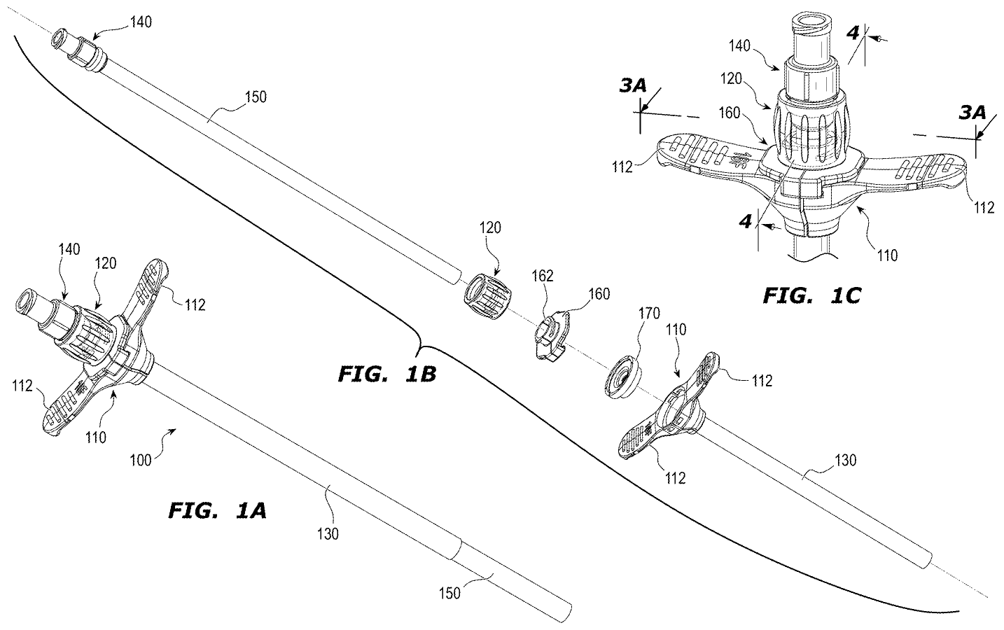

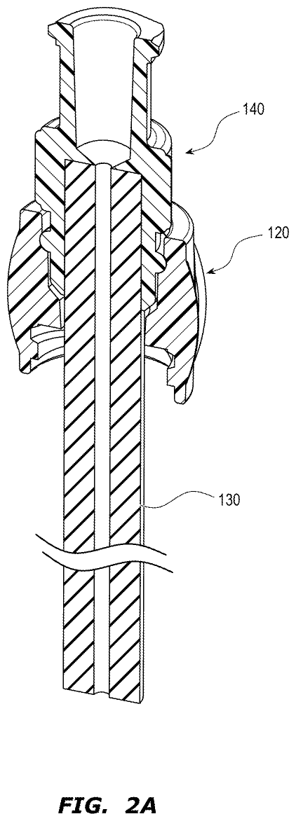

FIG. 1A is a perspective view of an introducer sheath assembly 100 having a dilator 140 coupled thereto, FIG. 1B is an exploded view of the introducer sheath assembly 100 and the dilator 140, FIG. 1C is a perspective view of a proximal portion of the introducer sheath assembly 100 having the dilator 140 coupled thereto, and FIG. 2 is a cross-sectional view of a portion of the dilator 140 and a shaft 150 of the dilator, according to various embodiments. While the introducer sheath assembly 100 is shown with the dilator 140 in FIGS. 1A-1C, the introducer sheath assembly 100 may be used with any medical instrument having an elongate member, such as dialysis catheters or other catheters.

The introducer sheath assembly 100 is configured to be split apart after insertion of the elongate member (e.g., shaft 150) for removal of the introducer sheath assembly 100 without disruption of the elongate member (e.g., shaft 150). For example, the introducer sheath assembly 100 may include a hub 110 comprising two arms 112, a cap 160 secured or securable to the hub 110, an elastomeric member 170 (e.g., valve) disposed at least partially within the hub 110, and a sheath 130 (e.g., introducer sheath) secured or securable to the hub 110. One or more (e.g., all) of the cap 160, the elastomeric member 170, the hub 110, and the sheath 130 may be configured to be spilt apart after insertion of the shaft 150 of the dilator 140 during use to allow the introducer sheath assembly 100 to be removed without disrupting the dilator 140 or other medical instrument having an elongate member. In some embodiments, a lock 120 (e.g., swivel lock) may selectively couple a coupling head 162 of the cap 160 to the dilator 140 or other medical instrument.

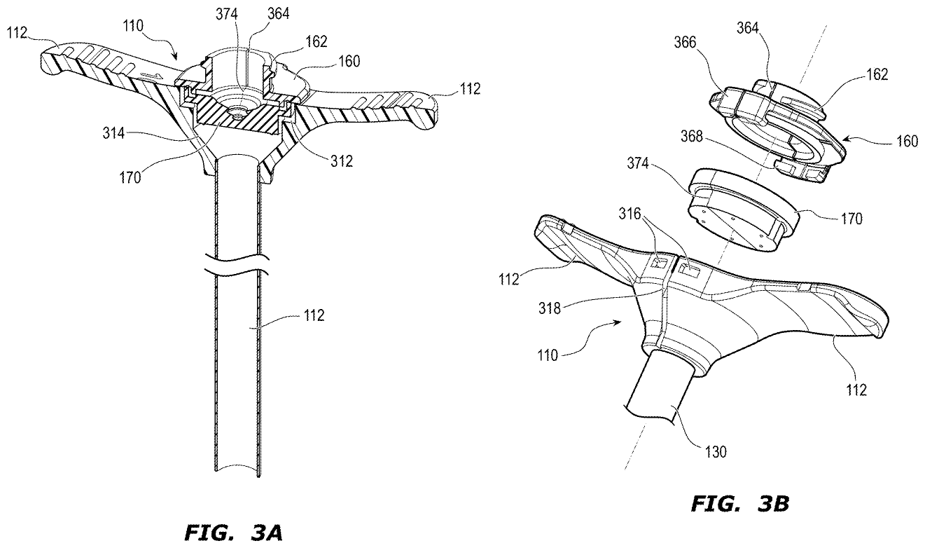

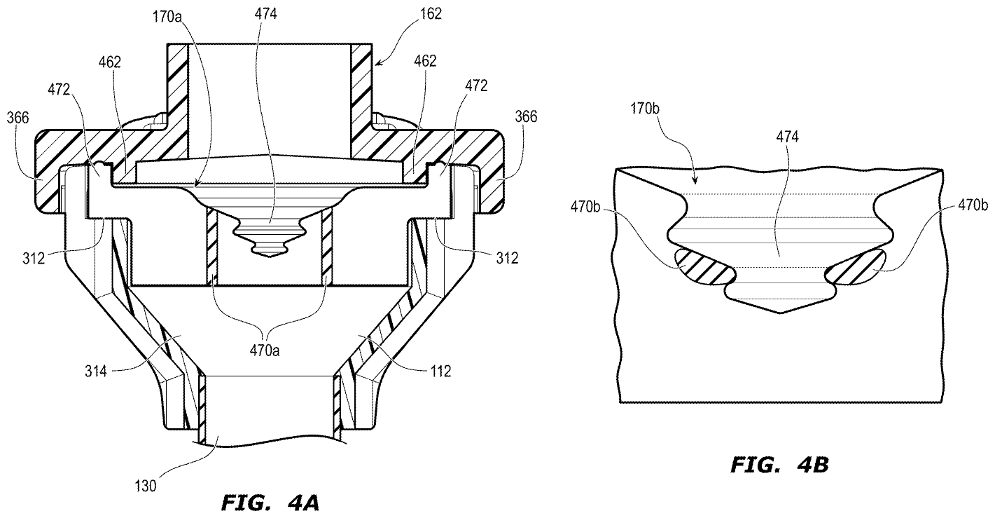

Turning ahead in the drawings, FIG. 3A is a cross-sectional view of the proximal portion of the introducer sheath assembly 100 taken through line 3-3 in FIG. 1C with the dilator 140 removed, FIG. 3B is an exploded view of the proximal portion of the introducer sheath assembly 100 in FIG. 1C with the dilator 140 removed, and FIG. 4A is a cross-sectional view of the proximal portion of the introducer sheath assembly 100 taken through line 4-4 in FIG. 1C with the dilator 140 removed.

The two arms 112 of the hub 110 may extend outward from a central region 314 of the hub 110. In some embodiments, the two arms 112 are disposed on the hub 110 substantially opposite of one another. Each arm of the two arms 112 may be ribbed to provide a gripping surface on each arm of the two arms 112.

The hub 110 may include a lumen 315 extending through the central region 314. The lumen 315 may include a substantially circular cross-sectional shape such that the lumen 315 is cylindrical, conical, frustoconical, or combinations thereof. In some embodiments, the lumen 315 narrows between cap 160 and the sheath 130. For example, the central region 314 of the hub 110 may an interior shelf 312 positioned within the lumen 315 a proximal end and a distal end of the lumen 315. In some embodiments, the interior shelf 312 is positioned to support at least a portion of the elastomeric member 170 thereon between the interior shelf 312 and the cap 160. The lumen 315 may further narrow or funnel between the interior shelf 312 and the distal end of the lumen 315.

The hub 110 includes two sidewalls 317 each extending at least partially between the two arms 112, according to an embodiment. Each sidewall of the two sidewalls 317 may include at least one (e.g., both) of a slot and/or a reduced thickness portion 318. The at least one of the slot and/or the reduced thickness portion 318 of a first sidewall of the two sidewalls 317 may be disposed substantially opposite to (e.g. 180 degrees from) the at least one of the slot and/or the reduced thickness portion 318 of a second sidewall of the two sidewall 317. In some embodiments, the at least one of the slot and/or the reduced thickness portion 318 of the two sidewalls 317 includes both (1) a slot comprising a gap extending from a proximal rim of the central region 314 of the hub 110 to the interior shelf 312 in each sidewall of the two sidewalls 317, and (2) a reduced thickness portion 318 extending distally from the interior shelf 312 in each sidewall of the two sidewalls 317. The reduced thickness portion 318 may extend from the interior shelf 312 to a distal, terminating end of the hub 110. The slot and/or the reduced-thickness portions 318 be configured to facilitate breaking or splitting of the hub 110 along the reduced thickness portions 318 of the two sidewalls 317. For example, the hub 110 may be configured to break into two separation portions or regions that are separated by the slot and/or the reduced-thickness portions 318 of the sidewalls 317.

The two arms 112 may be configured to facilitate splitting of the hub 110 (or components thereof, such as the cap 160 and/or the elastomeric member 170). Stated differently, the two arms 112 may be configured to separate portions of the hub 110 from one another to permit removal of the housing from an elongate member that extends through the elastomeric member 170 without retracting the hub 110 over the proximal end of the elongate member. For example, a practitioner may apply a first force to the central region 314 of the hub 110 and opposing forces on the two arms 112, thereby causing the hub 110 to break, snap, or otherwise split apart. In some embodiments, the hub 110 may break, snap, or otherwise split apart at the reduced thickness portion 318 of each sidewall of the two sidewalls 317, thereby breaking the hub 110 into two separate pieces.

The two sidewalls 317 of the hub 110 may define at least one opening 316 disposed at least proximate to the at least one of the slot and/or the reduced thickness portion 318. In some embodiments, the at least one opening 316 of each sidewall of the two sidewalls 317 includes two openings 316 in each sidewall of the two sidewalls 317. The at least one of the slot and/or the reduced thickness portion 318 in each sidewall of the two sidewalls 317 may extend between the two openings 316. In some embodiments, the gap of the slot is disposed between the two openings 316.

The elastomeric member 170 is disposed at least partially in the first lumen 315. The elastomeric member 170 of the introducer sheath assembly 100 may be configured to be at least partially disposed within the lumen 315 of the hub 110. The elastomeric member 170 may form a variable-width channel 474 that extends partway through the elastomeric member 170. The channel 474 may be centered around and extending along the longitudinal axis of the introducer sheath assembly 100. As described in further detail below in connection with other figures, the elastomeric member 170 may be configured to form a liquid-tight seal around an elongate member (not shown) that extends through the introducer sheath assembly 100.

The elastomeric member 170 may be shaped and sized to be disposed at least partially on interior shelf 312 and extend distally into the lumen 315 from the interior shelf 312. In some embodiments, the elastomeric member 170 may include a proximal lip 472, which may generally annular.

In some embodiments, the elastomeric member 170 is formed from silicone rubber or other deformable elastomeric material. The inner diameter defined by the hub 110 may radially compress the elastomeric member 170. Stated differently, when disposed within the inner diameter defined by the hub 110, the elastomeric member 170 may be compressed toward a longitudinal axis of the introducer sheath assembly 100.

The elastomeric member 170 may form a channel 474 that extends partway through the elastomeric member 170 when the introducer sheath assembly 100 is in the resting state (i.e., no elongate member is disposed across the elastomeric member 170). The elastomeric member 170 may include a plurality of inward-extending protrusions and a plurality of inner sealing surfaces. When the introducer sheath assembly 100 is in the resting state, the at least one of the inner sealing surfaces may have a diameter that corresponds to the outer diameter of an elongate member that is 9 F to 24 F, such as 14 F to 20 F. The elastomeric member 170 also may include one or more relief surfaces that form one or more relief spaces. The channel 474 may, at least in part, be defined by a plurality of inner surfaces (e.g., sealing surfaces and relief surfaces) of the elastomeric member 170.

As shown in FIGS. 3A-3B, a slit 374 may extend at least partway through the elastomeric member 170. For example, in some embodiments, the slit 374 extends at least from a distal end of the elastomeric member 170 to at least a distal end of the channel 474. In some embodiments, the slit 374 extends from a proximal end of the elastomeric member 170 to a distal end of the elastomeric member 170, thereby dividing the elastomeric member 170 into two regions interfacing one another at the slit 374. In some embodiments, the slit 374 is substantially aligned with the at least one of the slot and/or the reduced-thickness portion 318 of the hub 110. In some embodiments, the elastomeric member 170 is manufactured (e.g., molded) as a single piece and the cut into the two regions.

With specific reference to FIG. 4A, the elastomeric member 170 also may include one or more connecting regions 470a extending between the two regions of the elastomeric member 170. Stated another way, the slit 474 may extend through the entirety of the elastomeric member with the exception of the one or more connecting regions 470a. Thus, while hatching is used in the cross-sectional view provided in FIG. 3A, hatching is shown only in the connection regions 470a in FIG. 4A due to orientation of the views with respect to the slit 374.

In some embodiments, the one or more connecting regions 470a include two connecting regions 470a having the channel 474 extending at least partially therebetween. In some embodiments, the two connecting regions 470a extend from the variable-width channel 474 to a distal, terminating end of the elastomeric member 170. The two connection regions 470a may be substantially parallel to a longitudinal axis of the introducer sheath assembly 100. Turning to FIG. 4B, in some embodiments of an elastomeric member 170b, the two connection regions 470b may be extend only partially between the variable-width channel 474 and the distal, terminating end of the elastomeric member 170b.

An elastomeric member 170 that includes the two regions at least partially separated by the slit 474 may facilitate removal of the elastomeric member 170. For example, when a practitioner desires to uncouple the elastomer member 170 from around an elongate member that extends through the elastomeric member 170, the practitioner may break or split the hub 110 into two separate portions as described elsewhere herein. The two regions of the elastomeric member 170 may be held between the two unconnected portions of the cap 160 and the two separate portions of the hub 110 such that the force of breaking or splitting the hub 110 into two separate portions breaks the one or more connection regions 470a, 470b extending between the two regions of the elastomeric member 170. According the elastomeric member 170 also splits into two separate regions as the hub 110 is split or broken into two separate portions, according to an embodiment. Stated differently, the elastomeric member 170 may be made from material that allows the practitioner to tear the one or more connecting regions 470a, 470b once the hub 110 has been or removed or as the hub 110 is being split or broken into two separate portions, thereby allow removal of the elongate member from the torn or separated elastomeric member 170 without retracting the elastomeric member over the proximal end of the elongate member.

The introducer sheath assembly 100 also includes the cap 160 coupled to the hub 110 with the elastomeric member 170 disposed at least partially in the lumen 315. The cap 160 may include or define a lumen 365 extending therethrough and aligned with the channel 474 along the longitudinal axis of the introducer sheath assembly 100. In some embodiments, the cap 160 includes two substantially (e.g. entirely) unconnected portions 160a, 160b each at least partially defining the lumen 365 of the cap 160. The two unconnected portions 160a, 160b of the cap 160 may separate from one another as the hub 110 is split. The two unconnected portions 160a, 160b of the cap 160 may be generally aligned with the two regions of the elastomeric member 170 such that the at least one of the slot and/or the reduced thickness portion 318 of the two sidewalls 317 is disposed at least partially between the two unconnected portions 160a, 160b of the cap 160 and at least partially between the two regions of the elastomeric member 170.

The cap 160 may be secured or coupled to the hub 110 by adhesive, detents, and/or some other coupling mechanism, thereby securing the elastomeric member 170 at least partially within the lumen 315 of the hub 110. More particularly, in some embodiments, the cap 160 includes multiple tabs 366 comprising an inward oriented projection 368 (shown in FIG. 3B) extending into each opening 316 of the at least one opening in the hub 110. Each unconnected portion of the two connected portions 160a, 160b may include at least one tab of the multiple tabs 366. Even more particularly, the multiple tabs 366 may include two 366 tabs on each unconnected portion of the two unconnected portions 160a, 160b of the cap 160. For example, a first tab of the two tabs 366 on each unconnected portion of the two unconnected portions 160a, 160b of the cap 160 may comprise a first inward oriented projection 368 extending into an opening of the two openings 316 in a first sidewall of the two sidewalls 317, and a second tab of the two tabs 366 on each connected portion of the two unconnected portions 160a, 160b of the cap 160 may comprise a second inward oriented projection 368 extending into an opening of the two openings 316 in a second sidewall of the two sidewalls 317 such that the at least one of the slot and/or the reduced thickness portion 318 of the two sidewalls 317 is disposed at least partially between the two unconnected portions 160a, 160b of the cap 160. When the cap 160 is secured to the hub 110, the inward oriented projections 368 may engage the hub 110 through the openings 116 to secure the elastomeric member 170 at least partially in the lumen 315 of the hub 110 between the cap 160 and the interior shelf 312 of the hub 110.

In some embodiments, the cap 160 includes one or more protrusions 462 extending distally from the cap 160. The one or more protrusions 462 may, for example, form a ring radially spaced from the multiple tabs 366 and protruding or extending distally from the cap 160. The proximal lip 472 of the elastomeric member 170 and the proximal rim of the central region 317 of the hub 110 may be secured between the multiple tabs 366 and the proximal lip 462 of the cap 160.

The cap 160 also may include the coupling head 162. In some embodiments, each unconnected portion of the two unconnected portions 160a, 160b may include at least of the coupling head 162. The introducer sheath assembly 100 also may include a lock, such as the swivel lock 120 detachably secured or securable to the coupling head 162 of the cap 160. In some embodiments, the coupling head 162 includes threading and the swivel lock 120 (or other lock) is threadedly engaged and/or coupled to the swivel lock 120.

The introducer sheath assembly 100 also includes the sheath 130, according to an embodiment. The sheath 130 may be secured or securable to the hub 110 opposite to the cap 160. In some embodiments, the sheath defines a lumen aligned along the longitudinal axis of the introducer sheath assembly 100 with the lumen 315 of the hub 110, the lumen 365 of the cap 160, and the channel 374 of the elastomeric member 170. In some embodiments, the sheath 130 includes a scored line or a weakened region to facilitate splitting of the sheath 130. The scored line or the weakened region may be generally aligned with the slit 374 of the elastomeric member and the at least one of the slot and/or the reduced thickness portion 318 in each sidewall of the two sidewalls 317.

The introducer assembly 100 may be removed from around an elongate member by splitting, tearing, breaking, or otherwise separating portions of the introducer sheath assembly 100 from one another. For example, in the depicted embodiment, a practitioner may apply a first force to the central region 314 of the hub 110 and opposing forces on the arms 112 of the hub 110, thereby causing the hub 110 to break and/or snap. The two regions of the elastomeric member 170 and/or the unconnected portions 160a, 160b of the cap 160 may separate substantially simultaneous with the breaking and/or snapping of the hub 110. The practitioner also may split the sheath 130. For example, the practitioner may withdraw the sheath 130 from the patient and make a longitudinal cut along the length of the sheath 130. As noted above, in some instances, the sheath 130 includes a scored line or a weakened region to facilitate making of the longitudinal cut and/or tearing of the sheath 130. In some embodiments, the scored line or weakened region is aligned with the reduced-thickness portions 318 of the hub 110 to facilitate separation. Once (1) portions of the hub 110, elastomeric member 170, and/or cap 160 have been separated from one another (e.g., via splitting or breaking) and (2) the sheath 130 has been cut or separated along its length, the hub 110 of the introducer sheath assembly 100 and the sheath 130 may be removed from the elongate member without retracting such components over a proximal end of the elongate member.

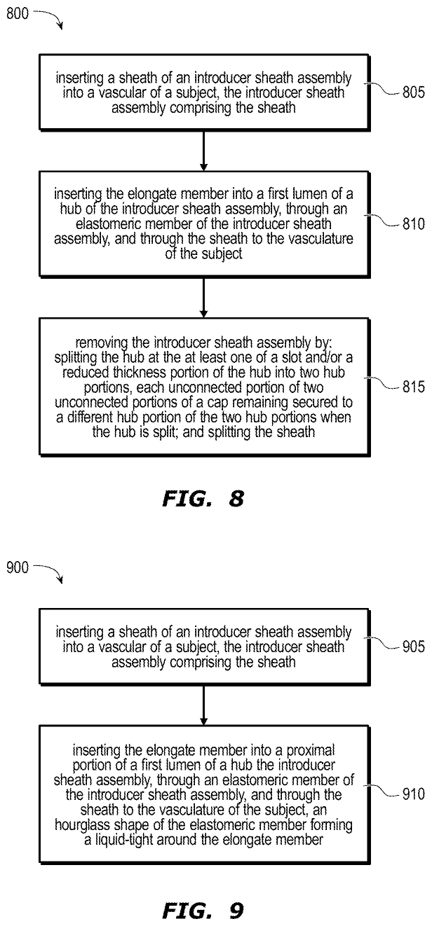

Turning ahead in the drawings, FIG. 8 is a flow diagram of a method 800 of inserting an elongate member into a subject. The method 800 may utilize any embodiments of the introducer sheath assembly 100. One or more steps shown in FIG. 8 may be optional and not necessarily included in each embodiment or process of the method 800. Further, various steps may be completed in difference sequences from those shown in FIG. 8.

In some embodiments, the method 800 includes coupling a lock to at least one of the coupling head of the cap and/or a portion of the elongate member.

The method 800 may include inserting 805 a sheath of an introducer sheath assembly into a vascular of a subject. The introducer sheath assembly may include the sheath, a hub, an elastomeric member, and a cap. The hub may comprise two arms extending outward from a central region of the hub, a first lumen extending through the central region, and two sidewalls each extending at least partially between the two arms and having at least one of a slot and/or a reduced thickness portion. The two sidewalls may define at least one opening disposed at least proximate to the at least one of the slot and/or the reduced thickness portion. The elastomeric member may be disposed at least partially in the first lumen. The cap may be coupled to the hub with the elastomeric member disposed at least partially in the first lumen. The cap may comprise a second lumen extending therethrough, multiple tabs comprising an inward oriented projection extending into each opening of the at least one opening in the hub, a coupling head extending away from the hub, and two unconnected portions each at least partially defining the second lumen, including at least one tab of the multiple tabs, and including at least some of the coupling head.

The method 800 also may include inserting 810 the elongate member into the first lumen of the hub, through the elastomeric member, and through the sheath to the vasculature of the subject. In some embodiments, the elongate member comprises an elongate instrument with a diameter of about 14 F to about 20 F and the elastomeric member forms a liquid-tight seal around the elongate instrument.

The method 800 also may include removing 815 the introducer sheath assembly. The introducer sheath may be removed by splitting the hub at the at least one of the slot and/or the reduced thickness portion into two hub portions and also splitting the sheath. The cap also may be split such that each unconnected portion of the two unconnected portions of the cap may remain secured to a different hub portion of the two hub portions when the hub is split.

In some embodiments of the method 800, the elastomeric member forms a channel that extends partially through the elastomeric member and two regions interfacing one another and connected by one or more connecting regions extending between the two regions. When the introducer sheath assembly is removed 815 by splitting the hub, each region of the two regions of the elastomeric member may remain secured to a different hub portion of the two hub portions.

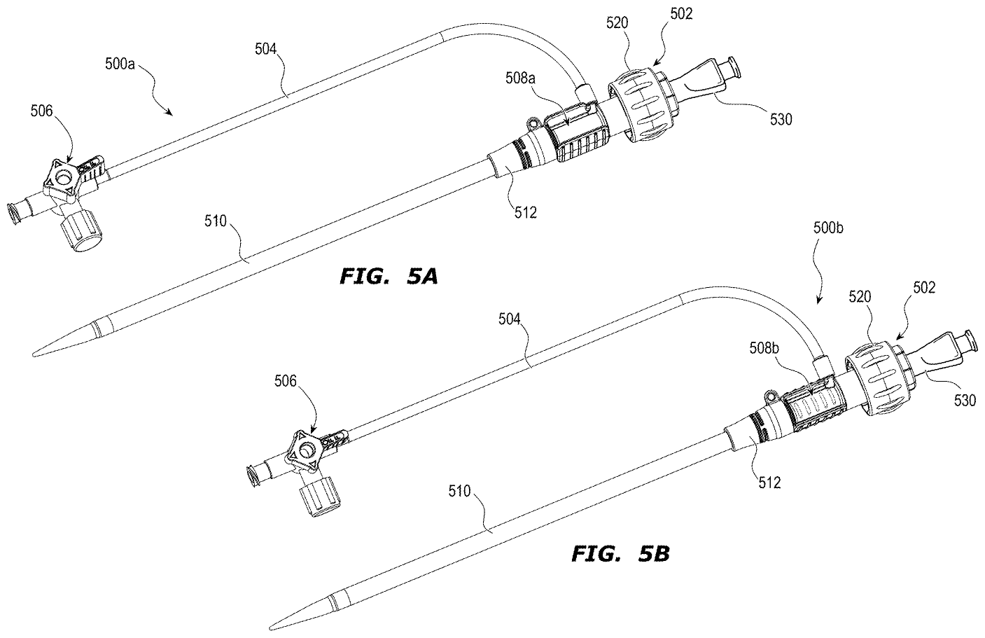

Turning now to FIGS. 5A-7C, in some embodiments, introducer sheath assemblies of this disclosure may be configured to include an elastomeric member that functions as an active valve within the introducer sheath assembly. For example, the elastomeric member of embodiments of introducer sheath assemblies of this disclosure may be selectively adjustable to increase, decrease, and/or close a lumen extending through the elastomeric member. This configuration of the elastomeric member may allow a single sized elastomeric member (and introducer sheath assembly) to be used with a variety of sizes of elongate members of medical instruments. In some embodiments, an elastomeric member of an introducer sheath assembly of this disclosure may be adjustable or otherwise configured to form a liquid-tight seal around any medical instrument having various sizes, including, for example, a size of 9 F to 16 F or 9 F to 20 F.

FIG. 5A is a perspective view of an introducer sheath assembly 500a and a dilator 530, according to one embodiment, and FIG. 5B is a perspective view of an introducer sheath assembly 500b and the dilator 530, according to one embodiment. The introducer sheath assembly 500a, 500b may include an introducer sheath 510, a hub 502, a cap 520 secured to a proximal region of the hub 502, a strain relief 512 secured to a distal region of the hub 502, a sidearm catheter 504, and a stopcock valve 506. In some embodiments the sidearm catheter 504 and the stopcock valve 506 are absent from the introducer sheath assembly 500a, 500b. While the hub 502 of the introducer sheath assembly 500a includes a wing grip member 508a, the hub 502 of the introducer sheath assembly 500b may include a hub grip 508b.

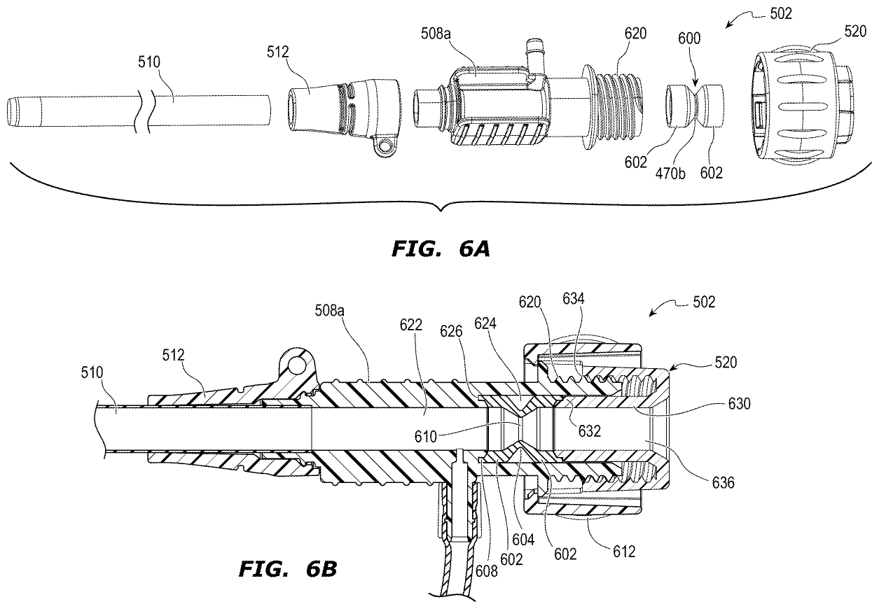

FIG. 6A is an exploded view of a proximal portion of the introducer sheath assembly 500a of FIG. 5A, and FIG. 6B is a cross-sectional view of the proximal portion of the introducer sheath assembly 500a of FIG. 5A. While FIGS. 6A-6B show the proximal portion of the introducer sheath assembly 500a, the introducer sheath assembly 500b of FIG. 5B may include any feature shown and described below in reference to the introducer sheath assembly 500a.

The hub 502 may include a proximal end region 620, a distal end region 621, and a lumen extending therethrough. The lumen may include at least a proximal portion 624 having a first width (e.g., diameter) and a distal portion 622 having a second width (e.g., diameter) that is less than the first width of the proximal portion 624 of the lumen in the hub 502. In some embodiments, the hub 502 includes an interior shelf comprising an annular recess 626 between the proximal portion 624 and the distal portion 622 of the lumen of the hub 502. The annular recess 626 may be disposed between an inner rim protruding proximally from the interior shelf.

In embodiments of the introducer sheath assembly 500a including the sidearm catheter 504, the sidearm catheter 504 may secured to the hub 502 at least proximate or near the distal portion 622 of the lumen such that the sidearm catheter 504 is in fluid communication with the lumen of the hub 502 via the distal portion 622. The introducer sheath 510 may be secured or securable to the distal end of the hub 502 such that a lumen of the introducer sheath 510 is in fluid communication with the distal portion 622 of the lumen of the hub 502.

The cap 520 may be coupled to the proximal end region 620 of the hub 502. In some embodiments, the cap 520 is configured a nut threadedly engaged with one or more threads 634 on the proximal end region 620 of the hub 502. The cap 520 may comprise an interior member 630 at least partially defining a lumen 636 and disposed within the proximal portion 624 of the lumen of the hub 502. The interior member 630 may include an interior wall or tube that is adjustably positioned or positionable within the proximal portion 624 of the lumen of the hub 502. The interior member 630 may include an annular recess 632 between an inner rim of the interior member 630 and the surface of the hub 502 defining the proximal portion 624 of the lumen of the hub 502.

In some embodiments, the cap 620 is adjustably secured to the proximal end region 620 of the hub 502 and configured to at least partially advance and/or at least partially withdraw the interior member 630 of the cap 520 from the proximal portion 624 of the lumen of the hub 502. For example, in embodiments in which the cap 520 is configured as a nut threadedly engaged to the proximal end region 620 of the hub 502, rotating the cap 520 a first direction may advance the interior member 630 in the proximal portion 624 of the lumen of the hub 502 and rotating the cap 520 in a second direction different than the first direction may at least partially withdraw the interior member 630 from the proximal portion 624 of the lumen of the hub 502.

The introducer sheath assembly also includes an elastomeric member 600 which may have a generally hourglass shape disposed in the proximal portion 624 of the lumen of the hub 502 between the interior member 630 of the cap 520 and the distal portion 622 of the lumen of the hub 502. The elastomeric member 600 comprises a lumen 610 extending therethrough and may be formed from silicone rubber or other deformable elastomeric material. In some embodiments, the elastomeric member 600 is configured to deform such that it conforms to an elongate instrument having a size of about 9 F to about 20 F being inserted through the lumen 610 of the elastomeric member 600 and form a liquid-tight seal around the elongate instrument.

In some embodiments, the elastomeric member 600 is selectively adjustable to at least decrease an interior width (e.g., diameter) of the lumen 610 and/or substantially close fluid communication between the lumen 636 of the cap 520 and the distal portion 622 of the lumen of the hub 502 responsive to advancement of the interior member 630 of the cap 520 further into the proximal portion 624 of the lumen of the hub 502. For example, in some embodiments, the elastomeric member 600 comprises two end regions 602, and outer surface, and a central region 604 disposed between the two end regions 602. In an undeformed state (shown in FIG. 6A) having no non-ambient forces exerted upon the elastomeric member 600, the elastomeric member 600 comprises the hourglass shape such that the central region 604 of the elastomeric member 600 comprising an inward oriented annular ridge in the lumen 610 of the elastomeric member and an annular groove in the outer surface of the elastomeric member 600.

The elastomeric member 600 may positioned in the proximal portion 624 of the lumen of the hub 502 such that advancing the interior member 630 of the cap 520 further into the proximal portion 624 of the lumen of the hub 502 decreases a distance between the two end regions 602 of the elastomeric member 600 and increases a height of the inward oriented annular ridge in the central region 604 of the elastomeric member 600. This increasing of the height of the inward oriented ridge in the central region 604 of the elastomeric member 600 decreases the interior width of the lumen 610 of the elastomeric member 600. As noted above, the cap 520 may be adjustably secured to the proximal end region 620 of the hub 502 and configured to advance the interior member 630 of the cap 520. In some embodiments, this advancement of the interior member 630 of the cap 520 may continue at least until the elastomeric member 600 is deformed such that the inward oriented annular ridge in the central region 604 of the elastomeric member 600 substantially closes the lumen 610 of the elastomeric member 600.

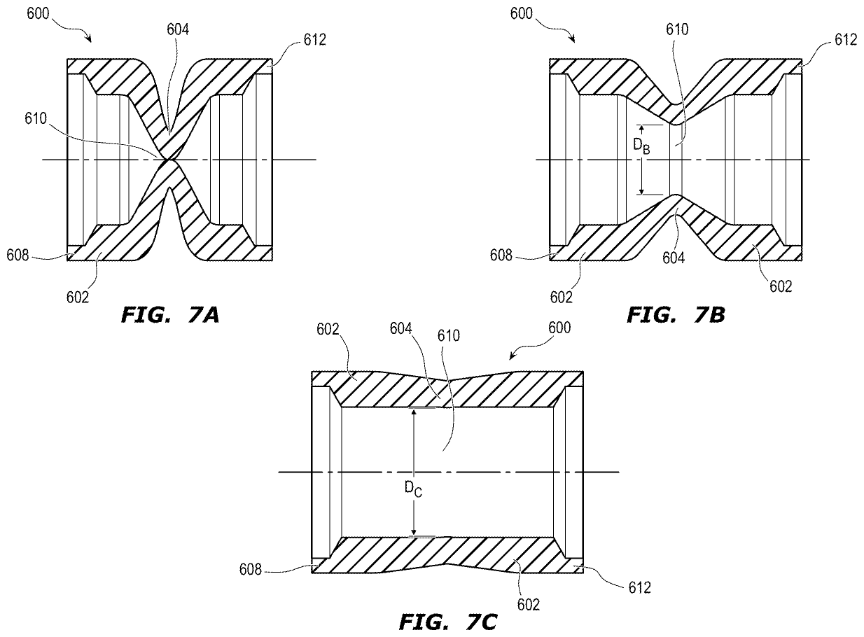

For example, a distal end region of the elastomeric member 600 may rest against the interior shelf of the hub 502 between the proximal portion 624 and the distal portion 622 of the lumen in the hub 502, and the interior member 630 of the cap 502 may then push against a proximal end region of the elastomeric member 600. In some embodiments, the distal end region of the elastomeric member 600 includes a distally protruding rim 608 that fits within the recess 608 of the interior shelf. In some embodiments, the proximal end region of the elastomeric member 600 includes a proximally protruding rim 612 (shown in FIGS. 7A-7C) that fits within the annular recess 632 between the inner rim of the interior member 630 and the surface of the hub 502 defining the proximal portion 624 of the lumen of the hub 502. These protruding rims of the elastomeric member 600 may prevent the elastomeric member 600 from slipping out of place as the elastomeric member 600 is compress by the interior member 630 of the cap 520.

Because the distal end region of the elastomeric member 600 is resting against the interior shelf of the hub 502, this force by the interior member 630 on the proximal end region of the elastomeric member 600 deforms the elastomeric member 600 by decreasing the distance between the proximal end region and the distal end region of the elastomeric member and forcing the inward oriented annular ridge in the central region 604 of the elastomeric member 600 to increase further inward. The inward oriented annular ridge in the central region 604 of the elastomeric member may be selectively deformed in such a manner until the inward oriented ridge contacts itself and substantially closes the lumen 610 of the elastomeric member 600.

In some embodiments, the elastomeric member 600 is secured to the cap 520 and the hub 502 such that the elastomeric member 600 deforms to decrease a height the inward oriented annular ridge as the interior member 630 of the cap 520 is at least partially withdrawn from the proximal portion 624 of the lumen of the hub 502, thereby increasing the interior width (e.g., diameter) of the lumen 610 of the elastomeric member 600. The elastomeric member 600 may be secured to the cap 520 and the hub 502 with one or more of an adhesive, ultrasonic welding, chemical bonding, and/or mechanical coupling.

In many embodiments, the elastomeric member 600 is configured to deform such that a height of the inward oriented annular ridge is decreased responsive to an elongate instrument being inserted through the lumen 610 of the elastomeric member 600, thereby forming a fluid tight-seal around the elongate instrument. For example, in an undeformed state (shown in FIG. 6A) having no non-ambient forces exerted upon the elastomeric member 600, the lumen 610 of the elastomeric member 600 may include a width or diameter of 9 F through the central region 604, and a width or diameter of 20 F through the end regions 602. When an elongate member having a size greater than 9 F, such as 14 F-20 F is inserted through the elastomeric member 600, elastomeric member 600 may deform such that the inward oriented ridge of the central region 604 is compressed or decreases to form a liquid-tight seal against any elongate member having a size greater than 9 F (such as 14 F-20 F).

FIGS. 7A-7C are cross-sectional views of the elastomeric member 600 of the introducer sheath assembly of FIG. 5A at various stages of deformation described above. For example, FIG. 7A shows the elastomeric member 600 in a compressed state from the interior member 630 of the cap 520 compressing the elastomeric member 600 against the interior shelf of the hub 502. In the compressed state shown in FIG. 7A, the elastomeric member 600 is compressed (e.g. deformed) such that the inward oriented ridge in the central region 604 substantially closes the lumen 610 extending through the elastomeric member 600.

FIG. 7B shows the elastomeric member 600 in a natural, undeformed state having no non-ambient forces exerted upon the elastomeric member 600. In the undeformed state shown in FIG. 7B, the lumen 610 of the elastomeric member 600 has a width or diameter of DB in the central region 604 of the lumen 610. Stated another way, DB may be the width or diameter of the central region 604 of the lumen 610 at the inward oriented ridge of the elastomeric member 600 and also the minimum or smallest width or diameter of the lumen 610 when the elastomeric member 600 is in the undeformed state. In the undeformed state having no non-ambient forces exerted upon the elastomeric member 600, the elastomeric member 600 retains the hourglass shape with the central region of the elastomeric member 600 having the inward oriented ridge and the annular groove on the outer surface. In some embodiments, in the undeformed state, DB in the central region 604 of the lumen is about 5 F to about 20 F, about 6 F to about 12 F, about 12 F to about 18 F, about 5 F to about 7 F, about 6 F to about 8 F, about 7 F to about 9 F, about 8 F to about 10 F, about 9 F to about 11 F, about 10 F to about 12 F, about 11 F to about 13 F, about 12 F to about 14 F, about 13 F to about 15 F, about 14 F to about 16 F, about 15 F to about 17 F, about 16 F to about 18 F, about 17 F to about 19 F, about 18 F to about 20 F, about 5 F, about 6 F, about 7 F, about 8 F, about 9 F, about 10 F, about 11 F, about 12 F, about 13 F, about 14 F, about 15 F, about 16 F, about 17 F, about 18 F, about 19 F, or about 20 F.

FIG. 7C shows the elastomeric member 600 deformed responsive to at least one of (1) an elongate member (not shown) having a width or diameter of Dc being inserted through and present in the lumen 610, or (2) the interior member 630 of the cap 520 being at least partially withdrawn from the proximal portion 624 of the lumen of the hub 502 when the elastomeric member 600 is secured to the hub 502 and the interior member 630 of the cap 520. In the deformed stated of the elastomeric member 600 shown in FIG. 7C, the lumen 610 includes a width or diameter of Dc in the central region 604. Stated another way, Dc may be the width or diameter of the central region 604 of the lumen 610 at the inward oriented ridge of the elastomeric member 600 and also the minimum or smallest width or diameter of the lumen 610 when the elastomeric member 600 is in a deformed state due to at least one of (1) the inward oriented ridge of the central region 604 being compressed by an elongated member having a width or diameter greater than DB, and/or (2) the elastomeric member 600 is stretched longitudinally to decrease or eliminate the inward oriented ridge of the central region 604. Generally, the width or diameter of Dc of the lumen 610 in the central region 604 in the deformed stated is greater than the width or diameter of DB of the lumen 610 in the central region 604 of the elastomeric member 600 in the undeformed state. In some embodiments, in the deformed state, Dc in the central region 604 of the lumen is about 10 to about 24 F, about 14 F to about 20 F, about 10 F to about 12 F, about 11 F to about 13 F, about 12 F to about 14 F, about 13 F to about 15 F, about 14 F to about 16 F, about 15 F to about 17 F, about 16 F to about 18 F, about 17 F to about 19 F, about 18 F to about 20 F, about 19 F to about 21 F, about 20 F to about 22 F, about 21 F to about 23 F, about 22 F to about 24 F. In some embodiments, a single elastomeric member 600 is configured to form a liquid-tight seal around elongate members having a variety of sizes. For example, the elastomeric member 600 may have a DB of less than 14 F (such about 9 F) in the undeformed state, and may be configured to accommodate any elongate members having a diameter or width of about 14 F to about 20 F, with the elastomeric member 600 deforming to a Dc of about 14 F to about 20 F complementary to the diameter or width of the elongate member.

Turning ahead in the drawings, FIG. 9 is a flow diagram of a method 900 of inserting an elongate member into a subject. The method 900 may utilize any embodiments of the introducer sheath assembly 500a, 500b. One or more steps shown in FIG. 9 may be optional and not necessarily included in each embodiment or process of the method 900. Further, various steps may be completed in difference sequences from those shown in FIG. 9.

In some embodiments, the method 900 includes inserting 905 a sheath of an introducer sheath assembly into a vascular of a subject. The introducer sheath assembly of the method 900 may comprise the sheath, a hub, a cap, and an elastomeric member. The hub may comprise a proximal end region, a distal end region, and a first lumen extending therethrough. The first lumen may include at least a proximal portion having a first width and a distal portion having a second width that is less than the first width. The cap may be coupled to the proximal end region of the hub and may comprise an interior member at least partially defining a second lumen and disposed within the proximal portion of the first lumen. The elastomeric member may have an hourglass shape disposed in the proximal portion of the first lumen between the interior member of the cap and the distal portion of the first lumen. The elastomeric member may comprise a third lumen extending therethrough.

The method 900 may include inserting 910 the elongate member into the proximal portion of the first lumen of the hub, through the elastomeric member, and through the sheath to the vasculature of the subject. The hourglass shape of the elastomeric member may form a liquid-tight seal around the elongate member. The elongate member may have a size of about 9 F to about 20 F, such as 14 F to 20 F.

In some embodiments, the method 900 may further comprise advancing the interior member of the cap further into proximal portion of the first lumen to decrease an interior width of the third lumen and/or substantially close fluid communication between the second lumen of the cap and the distal portion of the first lumen. Advancing the interior member of the cap further into the proximal portion of the first lumen may decrease a distance between two end regions of the elastomeric member and/or may increase a height of an inward oriented annular ridge of the elastomeric member, thereby decreasing the interior width of the third lumen. In some embodiments, advancing the interior member of the cap further into proximal portion of the first lumen may include advancing the interior member of the cap into the proximal portion of the first lumen until the elastomeric member is deformed such that the inward oriented annular ridge substantially closes the third lumen of the elastomeric member.

In some embodiments, the method 900 further comprises at least partially withdrawing the interior member of the cap from the proximal portion of the first lumen to increase the interior width of the third lumen. In some embodiments, the cap may comprise a nut threadedly engaged with the proximal end region of the hub.

References to approximations are made throughout this specification, such as by use of the term “near.” For each such reference, it is to be understood that, in some embodiments, the value, feature, or characteristic may be specified without approximation. For example, where qualifiers such as “near” and “approximately” are used, these terms include within their scope the qualified words in the absence of their qualifiers. For example, where the term “approximately aligned” is recited with respect to a feature, it is understood that in further embodiments, the feature can have a precisely aligned configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An introducer sheath assembly, comprising:
a hub comprising a proximal end region, a distal end region, and a first lumen extending therethrough, the first lumen including at least a proximal portion having a first width and a distal portion having a second width that is less than the first width;
a cap coupled to the proximal end region of the hub and comprising an interior member at least partially defining a second lumen and disposed within the proximal portion of the first lumen;
an elastomeric member having an hourglass shape disposed in the proximal portion of the first lumen between the interior member of the cap and the distal portion of the first lumen, wherein the elastomeric member comprises a third lumen extending therethrough; and
an introducer sheath secured or securable to the distal end region of the hub.

2. The introducer sheath assembly of claim 1, wherein the elastomeric member is selectively adjustable to at least decrease an interior width of the third lumen and/or substantially close fluid communication between the second lumen of the cap and the distal portion of the first lumen responsive to advancement of the interior member of the cap further into the proximal portion of the first lumen.

3. The introducer sheath assembly of claim 2, wherein:
the elastomeric member comprises two end regions, and outer surface, and a central region disposed between the two end regions and comprising an inward oriented annular ridge in the third lumen and an annular groove in the outer surface of the elastomeric member when the elastomeric member is in an undeformed orientation;
the elastomeric member is positioned in the proximal portion of the first lumen such that advancing the interior member of the cap further into the proximal portion of the first lumen decreases a distance between the two end regions of the elastomeric member and increases a height of the inward oriented annular ridge, thereby decreasing the interior width of the third lumen.

4. The introducer sheath assembly of claim 3, wherein the cap is adjustably secured to the proximal end region of the hub and configured to advance the interior member of the cap and deform the elastomeric member until the inward oriented annular ridge substantially closes the third lumen of the elastomeric member.

5. The introducer sheath assembly of claim 3, wherein the elastomeric member is secured to the cap and the hub such that the elastomeric member deforms to decrease a height the inward oriented annular ridge as the interior member of the cap is at least partially withdrawn from the proximal portion of the first lumen, thereby increasing the interior width of the third lumen.

6. The introducer sheath assembly of claim 3, wherein the elastomeric member is configured to deform such that a height of the inward oriented annular ridge is decreased responsive to an elongate instrument being inserted through the third lumen of the elastomeric member, thereby forming a fluid tight-seal around the elongate instrument.

7. The introducer sheath assembly of claim 1, wherein the elastomeric member is configured to deform responsive to an elongate instrument having a size of about 9 F to about 20 F being inserted through the third lumen of the elastomeric member and form a liquid-tight seal around the elongate instrument.

8. The introducer sheath assembly of claim 1, wherein the cap comprises a nut threadedly engaged with the proximal end region of the hub.

9. A method of inserting an elongate member into a subject, the method comprising:
inserting a sheath of an introducer sheath assembly into a vascular of a subject, the introducer sheath assembly comprising the sheath and:
a hub comprising a proximal end region, a distal end region, and a first lumen extending therethrough, the first lumen including at least a proximal portion having a first width and a distal portion having a second width that is less than the first width;
a cap coupled to the proximal end region of the hub and comprising an interior member at least partially defining a second lumen and disposed within the proximal portion of the first lumen; and
an elastomeric member having an hourglass shape disposed in the proximal portion of the first lumen between the interior member of the cap and the distal portion of the first lumen, wherein the elastomeric member comprises a third lumen extending therethrough; and
inserting the elongate member into the proximal portion of the first lumen of the hub, through the elastomeric member, and through the sheath to the vasculature of the subject, the hourglass shape of the elastomeric member forming a liquid-tight seal around the elongate member.

10. The method of claim 9, further comprising advancing the interior member of the cap further into proximal portion of the first lumen to decrease an interior width of the third lumen and/or substantially close fluid communication between the second lumen of the cap and the distal portion of the first lumen.

11. The method of claim 10, wherein advancing the interior member of the cap further into the proximal portion of the first lumen decreases a distance between two end regions of the elastomeric member and increases a height of an inward oriented annular ridge of the elastomeric member, thereby decreasing the interior width of the third lumen.

12. The method of claim 11, wherein advancing the interior member of the cap further into proximal portion of the first lumen includes advancing the interior member of the cap into the proximal portion of the first lumen until the elastomeric member is deformed such that the inward oriented annular ridge substantially closes the third lumen of the elastomeric member.

13. The method of claim 11, further comprising at least partially withdrawing the interior member of the cap from the proximal portion of the first lumen to increase the interior width of the third lumen.

14. The method of claim 9, wherein the elongate member has a size of about 9 F to about 20 F.

15. The method of claim 9, wherein the cap comprises a nut threadedly engaged with the proximal end region of the hub.
